(19) United States
(12) Patent Application Publication
(10) Pub. No.: US 2020/0155228 A1
(43) Pub. Date: May 21, 2020

(54) SHEATH DETECTION USING LOCAL IMPEDANCE INFORMATION

(71) Applicant: Boston Scientific Scimed Inc., Maple Grove, MN (US)

(72) Inventors: Leili Salehi, Waltham, MA (US); Alireza Ghodrati, Hopkinton, MA (US); Jacob I. Laughner, St. Paul, MN (US); Mordechai Perlman, Cambridge, MA (US)

(21) Appl. No.: 16/686,591

(22) Filed: Nov. 18, 2019

Related U.S. Application Data
(60) Provisional application No. 62/770,145, filed on Nov. 20, 2018.

(51) Int. Cl. A61B 18/14; A61M 25/00

(52) U.S. Cl. CPC A61B 18/1492; A61M 25/0074; A61B 2018/00892; A61B 2018/1467; A61M 25/0082

(57) ABSTRACT

A medical system including a catheter, a sheath, and a controller. The catheter having a catheter distal end that includes multiple electrodes, the sheath configured to receive the catheter and having a sheath distal end configured to cover one or more of the multiple electrodes, and the controller configured to supply a current between electrodes of the multiple electrodes and to measure voltages from at least two of the multiple electrodes to determine whether one or more of the multiple electrodes is covered by the sheath.

### CROSS-REFERENCE TO RELATED APPLICATION

This application claims priority to Provisional Application No. 62/770,145, filed Nov. 20, 2018, which is herein incorporated by reference in its entirety.

### TECHNICAL FIELD

The present disclosure relates to medical systems and methods for accessing locations and performing procedures within a patient's body. More specifically, the disclosure relates to systems and methods for determining the location of a sheath on a catheter.

### BACKGROUND

Catheters are often used in medical procedures to provide physical access to locations within a patient via a relatively small passageway, reducing the need for traditional invasive surgery. Catheters, such as cardiac catheters including electrodes, can be used for both diagnostic and therapeutic applications. Diagnostic applications include recording and mapping of the electrical signals generated by the heart. Therapeutic applications include cardiac pacing and ablation.

Ablation procedures are one of the most widely performed procedures for treatment of cardiac arrhythmias. In an ablation procedure, electrical energy, such as radio-frequency energy, is applied through one or more electrodes to the tissue of the patient's heart to form lesions in a desired portion of the patient's heart, for example the right atrium. When properly made, these lesions alter the conductive characteristics of portions of the patient's heart, thereby controlling the symptoms of arrhythmias, such as supraventricular tachycardia, ventricular tachycardia, atrial flutter, and atrial fibrillation. The success of an ablation procedure depends on the quality of the lesion or lesions created by the ablation catheter. To have a deep and effective lesion, the ablation catheter needs to be near the cardiac tissue. Ablation in blood pools can cause blood coagulation increasing the safety risk and decreasing the success rate of the ablation procedure.

A catheter sheath is often used to guide a catheter into a patient's body and position the catheter in the patient's body. In ablation procedures, a sheath can be used to guide the catheter between heart chambers and facilitate movement of the catheter inside each chamber. Also, a sheath can be used as an auxiliary lever to hold the catheter straight and in place when force is being exerted on the catheter.

To accurately place the catheter near the patient's cardiac tissue in an ablation procedure, the proximity of the catheter to the cardiac tissue is evaluated. Often, this evaluation includes using electrodes for measuring impedance and/or force sensors for measuring the force exerted by the catheter on the cardiac tissue. However, if the electrodes or the force sensors are covered by a sheath, which is usually a high impedance rigid object, the measurements lead to inaccurate estimations, such as under-estimations, of the proximity of the catheter to the cardiac tissue. This can result in poor placement of the ablation catheter and reduce the probability of success of the ablation procedure.

### SUMMARY

In an Example 1, a medical system, including a catheter having a catheter distal end that includes multiple electrodes, a sheath configured to receive the catheter and having a sheath distal end configured to cover one or more of the multiple electrodes, and a controller configured to supply a current between electrodes of the multiple electrodes and to measure voltages from at least two of the multiple electrodes to determine whether one or more of the multiples electrodes is covered by the sheath.

In an Example 2, the medical system of Example 1, wherein the controller is configured to source the current from any one of the multiple electrodes and sink the current at any other one of the multiple electrodes.

In an Example 3, the medical system of any of Examples 1 and 2, wherein the controller is configured to calculate impedance values to determine whether the one or more of the multiple electrodes is covered by the sheath.

In an Example 4, the medical system of Example 3, wherein the controller is configured to determine a difference between two of the voltages measured and divide the difference by a multiple of the current to calculate one of the impedance values.

In an Example 5, the medical system of any of Examples 3 and 4, wherein the controller is configured to compare the impedance values to one or more threshold values to determine whether the one or more of the multiple electrodes is covered by the sheath.

In an Example 6, the medical system of any of Examples 1-5, wherein the catheter is a linear ablation catheter and the multiple electrodes are longitudinally spaced apart at the catheter distal end.

In an Example 7, a method, including positioning a distal end of a catheter in a sheath that is configured to receive the catheter and cover electrodes at the distal end of the catheter, providing a current between the electrodes at the distal end of the catheter, measuring voltages from at least two of the electrodes to a reference voltage, and determining whether one or more of the electrodes is covered by the sheath based on the current and the voltages measured.

In an Example 8, the method of Example 7, wherein providing a current comprises sourcing the current from one of the electrodes and sinking the current at another one of the electrodes.

In an Example 9, the method of any of Examples 7 and 8, wherein determining whether one or more of the electrodes is covered by the sheath includes calculating at least one impedance value and comparing the at least one impedance value to one or more threshold values.

In an Example 10, the method of Example 9, wherein calculating at least one impedance value includes determining a difference between two of the voltages and dividing the difference by a multiple of the current.

In an Example 11, the method of any of Examples 7-10, wherein the catheter includes four electrodes spaced apart at the distal end of the catheter, the catheter including a first electrode that is a most distal electrode at the distal end of the catheter, a second electrode spaced apart from and proximal the first electrode, a third electrode spaced apart from and proximal the second electrode, and a fourth electrode spaced apart from and proximal the third electrode and wherein providing a current comprises sourcing the current from the first electrode and sinking the current at the fourth electrode.

In an Example 12, the method of Example 11, wherein to determine whether the fourth electrode is covered by the sheath, measuring voltages from at least two of the electrodes includes measuring voltages from the first electrode to the reference voltage to get a first voltage, from the second electrode to the reference voltage to get a second voltage, from the third electrode to the reference voltage to get a third voltage, and from the fourth electrode to the reference voltage to get a fourth voltage. And, determining whether one or more of the electrodes is covered by the sheath includes subtracting the first voltage from the fourth voltage to get a first result and dividing the first result by a first multiple of the current to get a first impedance value, subtracting the second voltage from the fourth voltage to get a second result and dividing the second result by a second multiple of the current to get a second impedance value, subtracting the third voltage from the fourth voltage to get a third result and dividing the third result by a third multiple of the current to get a third impedance value, comparing the first impedance value to a first threshold value, comparing the second impedance value to a second threshold value, and comparing the third impedance value to a third threshold value.

In an Example 13, the method of Example 11, wherein to determine whether the third electrode is covered by the sheath, measuring voltages from at least two of the electrodes includes measuring voltages from the first electrode to the reference voltage to get a first voltage, from the second electrode to the reference voltage to get a second voltage, and from the third electrode to the reference voltage to get a third voltage. And, determining whether one or more of the electrodes is covered by the sheath includes subtracting the first voltage from the third voltage to get a first result and dividing the first result by a first multiple of the current to get a first impedance value, subtracting the second voltage from the third voltage to get a second result and dividing the second result by a second multiple of the current to get a second impedance value, comparing the first impedance value to a first threshold value, and comparing the second impedance value to a second threshold value.

In an Example 14, the method of Example 11, wherein to determine whether the second electrode is covered by the sheath, measuring voltages from at least two of the electrodes includes measuring voltages from the first electrode to the reference voltage to get a first voltage, and from the second electrode to the reference voltage to get a second voltage. And determining whether one or more of the electrodes is covered by the sheath includes subtracting the first voltage from the second voltage to get a first result and dividing the first result by a first multiple of the current to get a first impedance value, and comparing the first impedance value to a first threshold value.

In an Example 15, the method of Example 11, wherein to determine whether the first electrode is covered by the sheath, measuring voltages from at least two of the electrodes includes measuring voltages from the first electrode to the reference voltage to get a first voltage, from the second electrode to the reference voltage to get a second voltage, from the third electrode to the reference voltage to get a third voltage, and from the fourth electrode to the reference voltage to get a fourth voltage. And, determining whether one or more of the electrodes is covered by the sheath includes subtracting the second voltage from the first voltage to get a first result and dividing the first result by a first multiple of the current to get a first impedance value, subtracting the third voltage from the first voltage to get a second result and dividing the second result by a second multiple of the current to get a second impedance value, subtracting the fourth voltage from the first voltage to get a third result and dividing the third result by a third multiple of the current to get a third impedance value, comparing the first impedance value to a first threshold value, comparing the second impedance value to a second threshold value, and comparing the third impedance value to a third threshold value.

In an Example 16, a medical system, includes a catheter having a catheter distal end and including multiple electrodes situated at the catheter distal end, and a sheath having a sheath distal end and a lumen configured to receive the catheter such that the catheter distal end protrudes from the sheath distal end. The sheath configured to cover one or more of the multiple electrodes. Also, the medical system includes a controller configured to provide a current between electrodes of the multiple electrodes and measure at least two voltages from at least two of the multiple electrodes to a reference voltage to determine whether one or more of the multiples electrodes is covered by the sheath.

In an Example 17, the medical system of Example 16, wherein the controller is configured to source the current from any one of the multiple electrodes and sink the current at any other one of the multiple electrodes.

In an Example 18, the medical system of Example 16, wherein the controller is configured to measure a voltage from each of the multiple electrodes to the reference voltage to determine whether the one or more of the multiples electrodes is covered by the sheath.

In an Example 19, the medical system of Example 16, wherein the controller is configured to calculate impedance values between electrodes of the multiple electrodes to determine whether the one or more of the multiple electrodes is covered by the sheath.

In an Example 20, the medical system of Example 19, wherein the controller is configured to compare the impedance values calculated to one or more threshold values to determine whether the one or more of the multiple electrodes is covered by the sheath.

In an Example 21, the medical system of Example 16, wherein the controller is configured to calculate impedance values between electrodes of the multiple electrodes by determining a difference between the at least two voltages measured and dividing the difference by a multiple of the current provided via the controller.

In an Example 22, the medical system of Example 16, wherein the multiple electrodes are longitudinally spaced apart at the catheter distal end.

In an Example 23, the medical system of Example 16, wherein the catheter is a linear ablation catheter.

In an Example 24, a method that includes positioning a distal end of a catheter through a lumen of a sheath that is configured to cover electrodes at the distal end of the catheter. The distal end of the catheter including the electrodes configured to protrude from a distal end of the sheath. The method further including providing a current between electrodes at the distal end of the catheter, measuring voltages from each of at least two of the electrodes to a reference voltage, and determining whether one or more of the electrodes is covered by the sheath based on the current and the voltages measured.

In an Example 25, the method of Example 24, wherein providing a current comprises sourcing the current from one of the electrodes and sinking the current at another one of the electrodes.

In an Example 26, the method of Example 24, wherein providing a current comprises sourcing the current from a most distal one of the electrodes and sinking the current at a most proximal one of the electrodes.

In an Example 27, the method of Example 24, wherein determining whether one or more of the electrodes is covered by the sheath includes calculating at least one impedance value and comparing the at least one impedance value to one or more threshold values.

In an Example 28, the method of Example 24, wherein determining whether one or more of the electrodes is covered by the sheath includes calculating at least one impedance value by determining a difference between two of the voltages and dividing the difference by a multiple of the current.

In an Example 29, a method includes positioning a sheath over a catheter that includes four electrodes spaced apart at a distal end of the catheter. The catheter including a first electrode that is a most distal electrode at the distal end of the catheter, a second electrode spaced apart from and proximal the first electrode, a third electrode spaced apart from and proximal the second electrode, and a fourth electrode spaced apart from and proximal the third electrode. The distal end of the catheter configured to protrude from a distal end of the sheath that is configured to cover at least up to all four of the four electrodes. The method further including providing a current between any two of the four electrodes, measuring voltages from at least two of the four electrodes to a reference voltage, and determining whether one or more of the four electrodes is covered by the sheath.

In an Example 30, the method of Example 29, wherein providing a current comprises sourcing the current from the first electrode and sinking the current at the fourth electrode.

In an Example 31, the method of Example 29, wherein to determine whether the fourth electrode is covered by the sheath, measuring voltages from at least two of the four electrodes includes measuring voltages from the first electrode to the reference voltage to get a first voltage, from the second electrode to the reference voltage to get a second voltage, from the third electrode to the reference voltage to get a third voltage, and from the fourth electrode to the reference voltage to get a fourth voltage. The method further wherein determining whether one or more of the four electrodes is covered by the sheath includes subtracting the first voltage from the fourth voltage to get a first result and dividing the first result by a first multiple of the current to get a first impedance value, subtracting the second voltage from the fourth voltage to get a second result and dividing the second result by a second multiple of the current to get a second impedance value, subtracting the third voltage from the fourth voltage to get a third result and dividing the third result by a third multiple of the current to get a third impedance value, comparing the first impedance value to a first threshold value, comparing the second impedance value to a second threshold value, and comparing the third impedance value to a third threshold value.

In an Example 32, the method of Example 29, wherein to determine whether the third electrode is covered by the sheath, measuring voltages from at least two of the four electrodes includes measuring voltages from the first electrode to the reference voltage to get a first voltage, from the second electrode to the reference voltage to get a second voltage, and from the third electrode to the reference voltage to get a third voltage. The method further wherein determining whether one or more of the four electrodes is covered by the sheath includes subtracting the first voltage from the third voltage to get a first result and dividing the first result by a first multiple of the current to get a first impedance value, subtracting the second voltage from the third voltage to get a second result and dividing the second result by a second multiple of the current to get a second impedance value, comparing the first impedance value to a first threshold value, and comparing the second impedance value to a second threshold value.

In an Example 33, the method of Example 29, wherein to determine whether the second electrode is covered by the sheath, measuring voltages from at least two of the four electrodes includes measuring voltages from the first electrode to the reference voltage to get a first voltage and from the second electrode to the reference voltage to get a second voltage. The method further wherein determining whether one or more of the four electrodes is covered by the sheath includes subtracting the first voltage from the second voltage to get a first result and dividing the first result by a first multiple of the current to get a first impedance value, and comparing the first impedance value to a first threshold value.

In an Example 34, the method of Example 29, wherein to determine whether the first electrode is covered by the sheath, measuring voltages from at least two of the four electrodes comprises measuring voltages from the first electrode to the reference voltage to get a first voltage, from the second electrode to the reference voltage to get a second voltage, from the third electrode to the reference voltage to get a third voltage, and from the fourth electrode to the reference voltage to get a fourth voltage. The method further wherein determining whether one or more of the four electrodes is covered by the sheath includes subtracting the second voltage from the first voltage to get a first result and dividing the first result by a first multiple of the current to get a first impedance value, subtracting the third voltage from the first voltage to get a second result and dividing the second result by a second multiple of the current to get a second impedance value, subtracting the fourth voltage from the first voltage to get a third result and dividing the third result by a third multiple of the current to get a third impedance value, comparing the first impedance value to a first threshold value, comparing the second impedance value to a second threshold value, and comparing the third impedance value to a third threshold value.

In an Example 35, the method of Example 29, wherein positioning a sheath over a catheter comprises uncovering one or more of the four electrodes and covering up to all four of the four electrodes.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating a cardiac ablation system, according to embodiments of the disclosure.

FIG. 2 is a diagram illustrating the distal end of the catheter protruding from the sheath, according to embodiments of the disclosure.

FIG. 3 is a diagram illustrating the sheath covering the fourth electrode, according to embodiments of the disclosure.

FIG. 4 is a diagram illustrating graphs of the first, second, and third impedance values, respectively, according to embodiments of the disclosure.

FIG. 5 is a diagram illustrating the sheath covering the fourth electrode and the third electrode, according to embodiments of the disclosure.

FIG. 6 is a diagram illustrating graphs of the first and second impedance values, respectively, for determining whether the third electrode is covered by the sheath, according to embodiments of the disclosure.

FIG. 7 is a diagram illustrating the sheath covering the fourth electrode, the third electrode, and the second electrode, according to embodiments of the disclosure.

FIG. 8 is a diagram illustrating a graph of the first impedance value for determining whether the second electrode is covered by the sheath, according to embodiments of the disclosure.

FIG. 9 is a diagram illustrating the sheath covering the fourth electrode, the third electrode, the second electrode, and the first electrode, according to embodiments of the disclosure.

FIG. 10 is a diagram illustrating graphs of the first, second, and third impedance values, respectively, according to embodiments of the disclosure.

FIG. 11 is a diagram illustrating a method of determining which, if any, of the first, second, third, and fourth electrodes are covered by the sheath, according to embodiments of the disclosure.

FIG. 12 is a diagram further illustrating a method of determining which, if any, of the first, second, third, and fourth electrodes are covered by the sheath, according to embodiments of the disclosure.

FIG. 13 is a diagram illustrating a method for determining whether one or more electrodes on a catheter are covered by a sheath, and for determining the location of the sheath on the catheter, according to embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure include detecting and/or determining whether electrodes of a catheter are covered by a sheath based on local impedance values related to the electrodes of the catheter. In embodiments, these local impedance values are resistance values related to the electrodes of the catheter. Using local impedance values is a non-invasive and accurate way of determining whether the electrodes of the catheter are covered by the sheath. By determining and knowing whether the electrodes of the catheter are covered by the sheath, medical personnel can more accurately place the catheter in the patient's body, such as in the patient's heart, and improve the probability of success of the medical procedures. In addition, determining local impedance values does not interfere with other features of the medical system.

Embodiments of the disclosure include detecting and/or determining whether electrodes of an ablation catheter are covered by a sheath, such that by determining and knowing whether the electrodes of the ablation catheter are covered by the sheath, medical personnel can more accurately place the ablation catheter, and improve the quality of lesions and the probability of success of the ablation procedure. In embodiments, the ablation catheter is a linear ablation catheter.

In embodiments, a catheter has a distal end that includes electrodes longitudinally spaced apart from a distal tip to more proximal positions on the distal end of the catheter. The catheter including the electrodes is slid through a lumen of the sheath, such that the distal end of the catheter protrudes from a distal end of the sheath. The catheter and the sheath are moved in relation to one another such that the sheath may cover one or more of the electrodes and/or all of the electrodes at the distal end of the catheter are uncovered. Impedance values are determined based on current that flows between two or more of the electrodes and voltage measurements from the electrodes. These impedance values are compared to one or more thresholds to determine which electrodes are uncovered and covered by the sheath. In embodiments, these impedance values are resistance values compared to one or more thresholds to determine which electrodes are uncovered and covered by the sheath.

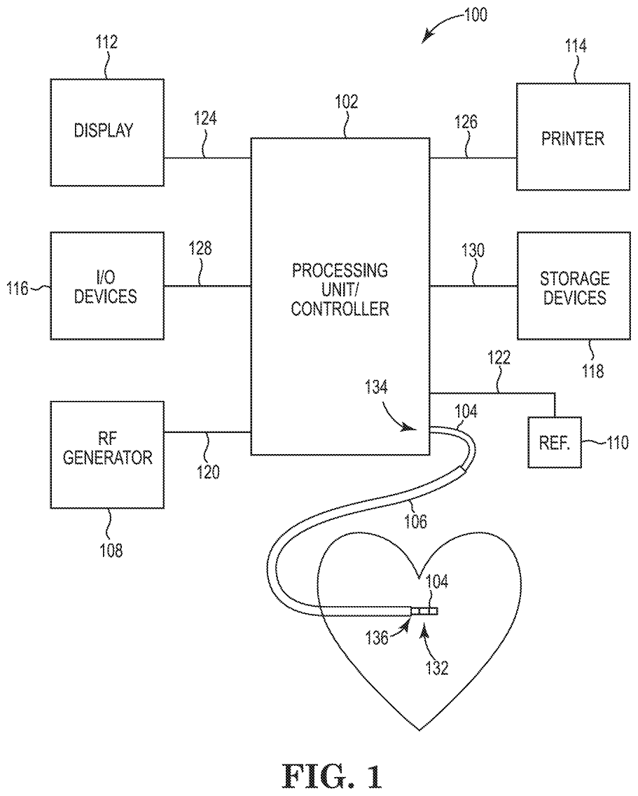

FIG. 1 is a diagram illustrating a cardiac ablation system 100, according to embodiments of the disclosure. The ablation system 100 includes a processing unit or controller 102, an ablation catheter 104, a catheter sheath 106, a radio frequency (RF) electrical signal generator 108, and an electrical reference 110, such as a patch on the patient or a ground line. In embodiments, the ablation catheter 104 is a linear ablation catheter.

In embodiments, the cardiac ablation system 100 also includes peripheral devices such as one or more displays 112, printers 114, input/output (I/O) devices 116, and storage devices 118, which are each communicatively coupled to the controller 102. In embodiments, the I/O devices 116 include one or more of a touchscreen, a keyboard, and/or a mouse, which are each communicatively coupled to the controller 102. While embodiments of the disclosure can be implemented in a cardiac ablation system, such as cardiac ablation system 100, other embodiments can be implemented in a cardiac mapping system, a recording system, a computer analysis system, and/or another suitable system.

The controller 102 is electrically coupled to the ablation catheter 104 to communicate with the ablation catheter 104, send signals such as alternating current (AC) and/or direct current (DC) signals to the ablation catheter 104, receive signals such as measured voltages from the ablation catheter 104, and to provide RF energy from the RF generator 108 to the ablation catheter 104. The controller 102 is electrically coupled to the RF generator 108 via one or more conductive paths 120 to communicate with the RF generator 108 and to provide the RF energy from the RF generator 108 to the ablation catheter 102 for ablating tissue. The controller 102 is electrically coupled to the electrical reference 110 via conductive path 122. In addition, in embodiments, the controller 102 is communicatively coupled to the displays 112 via communication paths 124, to the printers 114 via communication paths 126, to the I/O devices 116 via communication paths 128, and to the storage devices 118 via communication paths 130.

The controller 102 performs the functions and operations pertaining to ablation procedures and pertaining to detecting and determining whether electrodes of the ablation catheter 104 are covered by the sheath 106. In embodiments, the controller 102 includes one or more processors, micro-processors, and/or computers, which access software stored in memory to perform the functions and operations of the ablation system 100. In embodiments, the controller 102 includes one or more memory storage elements, such as volatile memory including one or more type of random access memory (RAM), non-volatile memory such as one or more type of read only memory (ROM), programmable memory, and/or a disc memory such as a hard drive memory, which store some or all of the software that is accessed by the controller 102 to perform the functions and operations of the ablation system 100. In embodiments, the storage devices 118 include one or more of volatile memory such as one or more type of random access memory (RAM), non-volatile memory such as one or more type of read only memory (ROM), programmable memory, and/or disc memory such as hard drive memory, which are each communicatively coupled to the controller 102 and which store some or all of the software that is accessed by the controller 102 to perform the functions and operations of the ablation system 100.

The ablation catheter 104 includes a flexible catheter body that carries one or more ablation electrodes, illustrated with lines in FIG. 1, at a distal end 132 of the ablation catheter 104. The one or more ablation electrodes are electrically coupled to the RF generator 108 that is configured to deliver ablation energy to the one or more ablation electrodes for ablating cardiac tissue. The ablation catheter 104 is movable, such that the ablation catheter 104 can be positioned with respect to the tissue to be treated. In other embodiments, the catheter 104 may be a mapping catheter, a diagnostic catheter, a CS catheter, and/or another suitable catheter. Where currents are provided to the catheter and voltage measurements are obtained from the catheter as described herein, and where subsequent processing of the current values and voltage measurements, as described herein, are performed in a corresponding system, such as a mapping system, a recording system, and/or another suitable system.

In embodiments, the catheter 104 includes the distal end 132 and a proximal end 134 coupled to the controller 102. The distal end 132 includes multiple electrodes longitudinally spaced apart at the distal end 132 of the catheter 104. In embodiments, the catheter 104 includes one electrode at the distal tip of the distal end 132 and one or more electrodes proximal the distal tip, where the one or more electrodes are longitudinally and proximally spaced apart at the distal end 132 of the catheter 104.

The catheter 104 including the electrodes is slid through a lumen of the sheath 106, such that the distal end 132 of the catheter 104 protrudes from a distal end 136 of the sheath 106. The catheter 104 and the sheath 106 are moved relative to one another to cover one or more of the electrodes at the distal end 132 of the catheter 104 with the distal end 136 of the sheath 106 or to uncover one or more of the electrodes at the distal end 132 of the catheter 104.

In embodiments, the controller 102 provides an AC current between two of the electrodes at the distal end 132 of the catheter 104 and the controller 102 measures voltages at one or more of the electrodes. The controller 102 then determines impedance values based on the AC current provided between the two electrodes and the voltage measurements. By comparing the impedance values to threshold values, the controller 102 determines whether one or more of the electrodes are covered by the sheath 106, i.e., the controller 102 determines the location or position of the catheter 104 in the sheath 106, which is the location or position of the sheath 106 on the catheter 104. By knowing whether the electrodes of the ablation catheter 104 are covered by the sheath 106, medical personnel can more accurately place the ablation catheter 104 near tissue being treated, which improves the quality of lesions and the probability of success of the ablation procedure. In some embodiments, the AC current has a constant root-mean-square (RMS) value. In some embodiments, the AC current has a constant peak-to-peak value.

In other embodiments, the controller 102 provides a constant DC current between two of the electrodes at the distal end 132 of the catheter 104 and the controller 102 measures voltages at one or more of the electrodes. The controller 102 then determines resistance values based on the DC current provided between the two electrodes and the voltage measurements. By comparing the resistance values to threshold values, the controller 102 determines whether one or more of the electrodes are covered by the sheath 106, i.e., the controller 102 determines the location or position of the catheter 104 in the sheath 106, which is the location or position of the sheath 106 on the catheter 104. By knowing whether the electrodes of the ablation catheter 104 are covered by the sheath 106, medical personnel can more accurately place the ablation catheter 104 near tissue being treated, which improves the quality of lesions and the probability of success of the ablation procedure.

In embodiments, the display devices 112 can display information pertaining to detecting and determining whether electrodes of the ablation catheter 104 are covered by the sheath 106. For example, the display devices 112 can display whether each of the electrodes is covered by the sheath or graphically display depictions of the location of the sheath 106 on the catheter 104 or over the electrodes of the catheter 104. Also, the display devices 112 can display data such as current value data, voltage measurement data, and the calculated impedance/resistance values for each of the procedures described herein. In some embodiments, the display devices 112 include one or more touchscreen displays for inputting and displaying data.

The illustrative cardiac ablation system 100 shown in FIG. 1 is not intended to suggest any limitation as to the scope of use or functionality of embodiments of the present disclosure. Neither should the illustrative cardiac ablation system 100 be interpreted as having any dependency or requirement related to any single component or combination of components illustrated herein. Additionally, various components depicted in FIG. 1 may be, in embodiments, integrated with various ones of the other components depicted herein and/or with components not illustrated, all of which are considered to be within the ambit of the subject matter disclosed herein. Additionally, or alternatively, aspects of embodiments of the cardiac ablation system 100 may be implemented in a computer analysis system configured to receive information from a controller and/or a memory device, e.g., a cloud server, and perform aspects of embodiments of the system described herein.

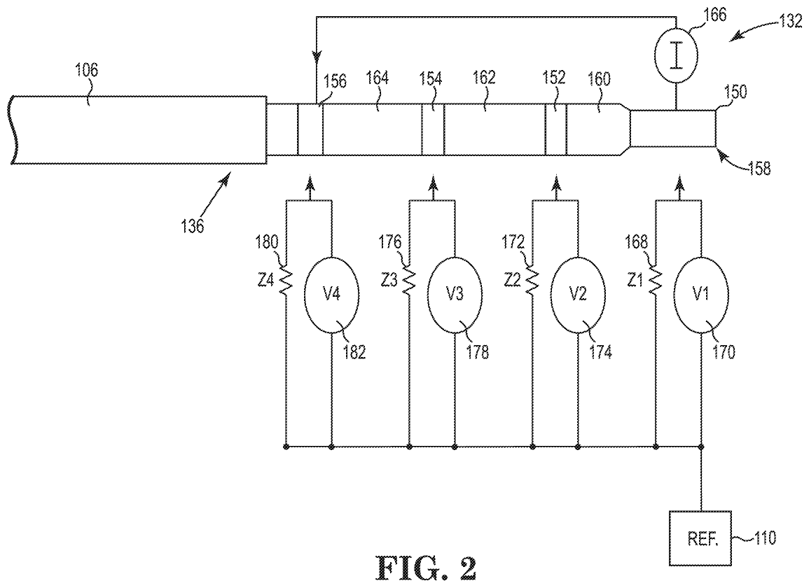

FIG. 2 is a diagram illustrating the distal end 132 of the catheter 104 protruding from the sheath 106, according to embodiments of the disclosure. The distal end 132 of the catheter 104 includes electrodes 150, 152, 154, and 156 longitudinally spaced apart at the distal end 132 of the catheter 104. The catheter 104 includes the first electrode 150 at the distal tip 158 of the distal end 132, the second electrode 152 proximal the first electrode 150, the third electrode 154 proximal the second electrode 152, and the fourth electrode 156 proximal the third electrode 154, where the electrodes 150, 152, 154, and 156 are longitudinally and proximally spaced apart at the distal end 132 of the catheter 104. In embodiments, each of the electrodes 150, 152, 154, and 156 is electrically insulated from each of the other electrodes 150, 152, 154, and 156 by insulating elements 160, 162, and 164.

In the operation of detecting and determining whether one or more of the electrodes 150, 152, 154, and 156 are covered by the sheath 106, current I at 166 is provided by the controller 102 to the first electrode 150, such that the first electrode 150 sources the current I and the fourth electrode 156 sinks the current I. In embodiments, the current I at 166 is an AC current having a constant RMS value. In embodiments, the current I at 166 is an AC current having a constant peak-to-peak value. In some embodiments, the current I at 166 has a magnitude of 2.5 microamps. In embodiments, the current I at 166 is a constant DC current and, in some embodiments, the current I at 166 is a constant DC current of 2.5 microamps. In other embodiments, the current I at 166 is provided by the controller 102 to another one of the electrodes 150, 152, 154, and 156, which sources the current I. Also, in other embodiments, the current I at 166 is sunk by another one of the electrodes 150, 152, 154, and 156.

As the current I at 166 flows from the first electrode 150 to the fourth electrode 156, an electric field is generated, and voltages are generated at each of the electrodes 150, 152, 154, and 156 based on and in response to the electric field. The magnitudes of the voltages at the electrodes 150, 152, 154, and 156 are related to the impedance of the surrounding medium. If a sheath, such as sheath 106, covers one of the electrodes 150, 152, 154, and 156, the sheath 106 creates a low conducting, high impedance cover, such that the impedance of the surrounding medium of the covered electrode increases and the measured voltage on the electrode increases. In FIG. 2 and in other figures of this disclosure, these impedances and voltages are depicted as one or more impedances extending from each of the electrodes 150, 152, 154, and 156 to the reference 110 and as a voltage from each of the electrodes 150, 152, 154, and 156 to the reference 110.

In FIG. 2, the sheath 106 is not covering any of the electrodes 150, 152, 154, and 156, and only one impedance is shown extending from each of the electrodes 150, 152, 154, and 156 to the reference 110. In FIG. 2, a first impedance 168 extends from the first electrode 150 to the reference 110 and a first voltage V1 170 is the voltage from the first electrode 150 to the reference 110, a second impedance 172 extends from the second electrode 152 to the reference 110 and a second voltage V2 174 is the voltage from the second electrode 152 to the reference 110, a third impedance 176 extends from the third electrode 154 to the reference 110 and a third voltage V3 178 is the voltage from the third electrode 154 to the reference 110, and a fourth impedance 180 extends from the fourth electrode 156 to the reference 110 and a fourth voltage V4 182 is the voltage from the fourth electrode 156 to the reference 110.

To determine whether one of the electrodes 150, 152, 154, and 156 is covered by the sheath 106, voltages at the electrodes 150, 152, 154, and 156 are measured and differential voltages from a single pair of the electrodes 150, 152, 154, and 156 or from multiple pairs of the electrodes 150, 152, 154, and 156 are determined. Each of these differential voltages is then divided by a multiple of the current I to determine a corresponding impedance value.

This can be shown as Zij=(Vᵢ−Vⱼ)/aI, where: the variable i does not equal the variable j; each of i and j is a number from 1 up to the number of electrodes, in this example 4; Zij is the calculated impedance value for a pair of electrodes; each of Vi and Vj is a voltage from one electrode of the pair of electrodes; and “a” is a constant that is multiplied by the current I. In embodiments, the multiple of the current I is the current I multiplied by the constant “a” that may or may not be an integer value. In embodiments, the constant “a” varies from one impedance calculation to another based on the physical distance between the pair of electrodes whose voltages are being used in the impedance calculation.

The calculated impedance values for an uncovered electrode are smaller than the calculated impedance values for the same electrode covered by the sheath 106. These calculated impedance values are compared to one or more threshold values to determine whether the electrode of interest is uncovered or covered by the sheath 106.

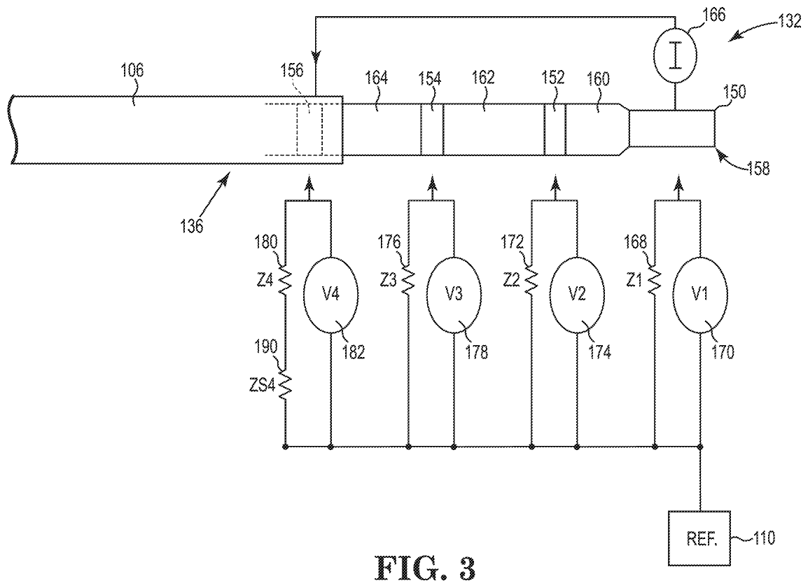

FIG. 3 is a diagram illustrating the sheath 106 covering the fourth electrode 156, according to embodiments of the disclosure. The distal end 132 of the catheter 104 protrudes from the sheath 106 such that the fourth electrode 156 is covered by the sheath 106 and the remaining electrodes 150, 152, and 154 are uncovered or not covered by the sheath 106. The sheath 106 creates a low conducting, high impedance cover over the fourth electrode 156, which increases the impedance from the fourth electrode 156 to the reference 110 and which increases the measured fourth voltage V4 182 on the fourth electrode 156. The increase in the impedance from the fourth electrode 156 to the reference 110 is depicted in FIG. 3 with the addition of series impedance ZS4 190 between the fourth electrode 156 and the reference 110.

In operation, the current I at 166 is provided by the controller 102 to the first electrode 150, such that the first electrode 150 sources the current I and the fourth electrode 156 sinks the current I. As the current I at 166 flows from the first electrode 150 to the fourth electrode 156, an electric field is generated, and voltages are generated at each of the electrodes 150, 152, 154, and 156 based on the electric field. Since the fourth electrode 156 is covered by the sheath 106, the fourth voltage V4 182 increases in comparison to when the fourth electrode 156 is uncovered.

To determine whether the fourth electrode 156 is covered or uncovered by the sheath 106, three impedance values are determined, one impedance value for each of electrode pair 156 and 150, electrode pair 156 and 152, and electrode pair 156 and 154. In some embodiments, less than three impedance values are determined, such as any two impedance values from each of electrode pair 156 and 150, electrode pair 156 and 152, and electrode pair 156 and 154.

The voltages at each of the electrodes 150, 152, 154, and 156 are measured and the controller 102 determines the impedance values. A first impedance value is determined by subtracting the first voltage V1 170 from the fourth voltage V4 182 and dividing the result by a multiple of the current I. A second impedance value is determined by subtracting the second voltage V2 174 from the fourth voltage V4 182 and dividing the result by a multiple of the current I. A third impedance value is determined by subtracting the third voltage V3 178 from the fourth voltage V4 182 and dividing the result by a multiple of the current I. In embodiments, the multiplier “a” is the same for each of the three calculations. In some embodiments, the multiplier “a” is different for one or more of the three calculations.

To determine whether the fourth electrode 156 is covered or uncovered by the sheath 106, each of the three impedance values is compared to a threshold value. In embodiments, the first impedance value is compared to a first threshold value, the second impedance value is compared to a second threshold value, and the third impedance value is compared to a third threshold value. In some embodiments, the first, second, and third threshold values are the same value. In some embodiments, one or more of the first, second, and third threshold values are different from the other threshold value(s).

For the ablation system 100 to determine that the fourth electrode 156 is covered by the sheath 106, the first impedance value must be greater than the first threshold value, the second impedance value must be greater than the second threshold value, and the third impedance value must be greater than the third threshold value.

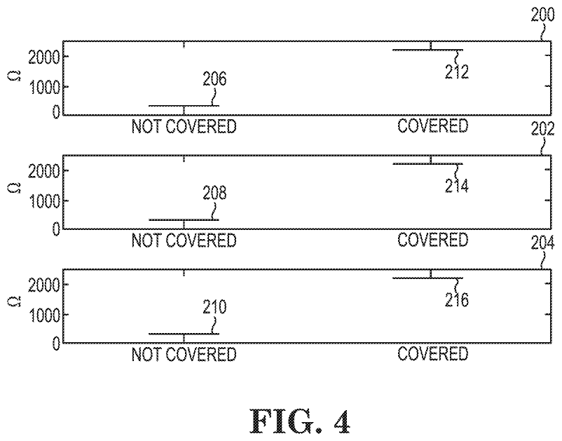

FIG. 4 is a diagram illustrating graphs 200, 202, and 204 of the first, second, and third impedance values, respectively, according to embodiments of the disclosure. Graph 200 shows the first impedance value, determined using the fourth voltage V4 182 and the first voltage V1 170, with the fourth electrode 156 uncovered and covered. Graph 202 shows the second impedance value, determined using the fourth voltage V4 182 and the second voltage V2 174, with the fourth electrode 156 uncovered and covered. Graph 204 shows the third impedance value, determined using the fourth voltage V4 182 and the third voltage V3 178, with the fourth electrode 156 uncovered and covered. In each of the graphs 200, 202, and 204, the y-axis represents the impedance value in ohms.

The uncovered impedance values 206, 208, and 210 shown in the graphs 200, 202, and 204, respectively, were calculated with the sheath 106 not covering any of the electrodes 150, 152, 154, and 156, as shown in FIG. 2. The voltages at each of the electrodes 150, 152, 154, and 156 were measured and the controller 102 determined the first uncovered impedance value 206, the second uncovered impedance value 208, and the third uncovered impedance value 210. The first uncovered impedance value 206 was determined by subtracting the first voltage V1 170 from the fourth voltage V4 182 and dividing the result by a multiple of the current I. The second uncovered impedance value 208 was determined by subtracting the second voltage V2 174 from the fourth voltage V4 182 and dividing the result by a multiple of the current I. The third uncovered impedance value 210 was determined by subtracting the third voltage V3 178 from the fourth voltage V4 182 and dividing the result by a multiple of the current I. In embodiments, the multiplier “a” is the same for each of the three calculations. In some embodiments, the multiplier “a” is different for one or more of the three calculations.

The covered impedance values 212, 214, and 216 shown in the graphs 200, 202, and 204, respectively, were calculated with the sheath 106 covering the fourth electrode 156 and not covering the other electrodes 150, 152, and 154, as shown in FIG. 3 and as described above in relation to FIG. 3.

The uncovered impedance values 206, 208, and 210 are smaller than the covered impedance values 212, 214, and 216. The first uncovered impedance value 206 is less than 1000 ohms and the first covered impedance value 212 is greater than 2000 ohms, such that a first threshold is set to be between the first uncovered impedance value 206 and the first covered impedance value 212 to distinguish between the uncovered and covered states of the fourth electrode 156 using the first impedance values 206 and 212. The second uncovered impedance value 208 is less than 1000 ohms and the second covered impedance value 214 is greater than 2000 ohms, such that a second threshold is set to be between the second uncovered impedance value 208 and the second covered impedance value 214 to distinguish between the uncovered and covered states of the fourth electrode 156 using the second impedance values 208 and 214. The third uncovered impedance value 210 is less than 1000 ohms and the third covered impedance value 212 is greater than 2000 ohms, such that a third threshold is set to be between the third uncovered impedance value 210 and the third covered impedance value 216 to distinguish between the uncovered and covered states of the fourth electrode 156 using the third impedance values 210 and 216.

The uncovered impedance values 206, 208, and 210 of less than 1000 ohms and the covered impedance values 212, 214, and 216 of greater than 2000 ohms in the graphs 200, 202, and 204 are example impedance values obtained using one catheter or one type of catheter. In other embodiments, using a different catheter or a different type of catheter, the uncovered impedance values 206, 208, and 210 of less than 1000 ohms may be different values and the covered impedance values of 212, 214, and 216 of greater than 2000 ohms may be different values and yet still distinguishable from one another, such that thresholds can be set between the uncovered impedance values and the covered impedance values as described above.

The covered impedance values are compared to the threshold values to determine whether the fourth electrode 156 is uncovered or covered by the sheath 106. In embodiments, all the covered impedance values 212, 214, and 216 must be greater than the corresponding threshold values for the controller 102 to make the determination that the fourth electrode 156 is covered by the sheath 106. In other embodiments, one or more of the covered impedance values 212, 214, and 216 must be greater than the corresponding threshold values for the controller 102 to make the determination that the fourth electrode 156 is covered by the sheath 106.

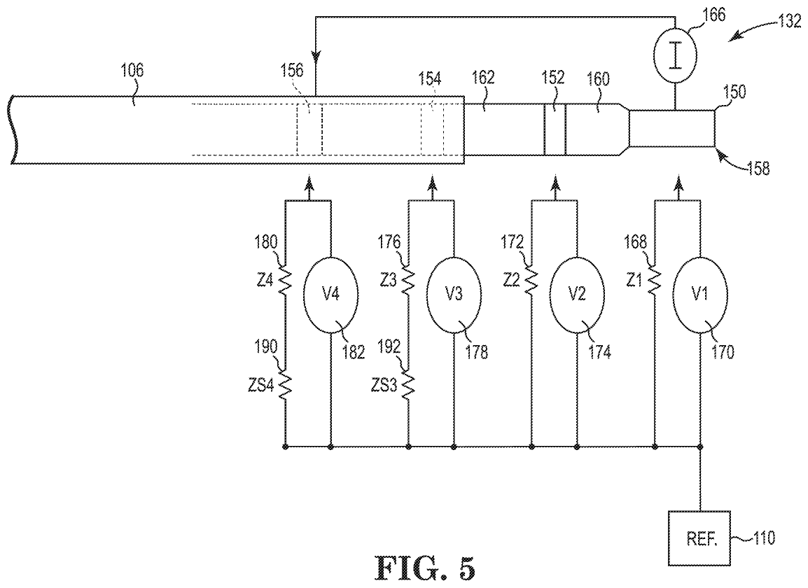

FIG. 5 is a diagram illustrating the sheath 106 covering the fourth electrode 156 and the third electrode 154, according to embodiments of the disclosure. In this situation, determining that the third electrode 154 is covered by the sheath 106, also determines that the fourth electrode 156 is covered by the sheath 106.

The distal end 132 of the catheter 104 protrudes from the sheath 106 such that the third and fourth electrodes 154 and 156 are covered by the sheath 106 and the remaining electrodes 150 and 152 are uncovered or not covered by the sheath 106. The sheath 106 creates a low conducting, high impedance cover over the third and fourth electrodes 154 and 156, which increases the impedance from the third electrode 154 to the reference 110 and the measured third voltage V3 178 on the third electrode 154, and which increases the impedance from the fourth electrode 156 to the reference 110 and the measured fourth voltage V4 182 on the fourth electrode 156. The increase in the impedance from the third electrode 154 to the reference 110 is depicted in FIG. 5 with the addition of series impedance ZS3 192 between the third electrode 154 and the reference 110 and the increase in the impedance from the fourth electrode 156 to the reference 110 is depicted with the series impedance ZS4 190 between the fourth electrode 156 and the reference 110.

In operation, the current I at 166 is provided by the controller 102 to the first electrode 150, such that the first electrode 150 sources the current I and the fourth electrode 156 sinks the current I. As the current I at 166 flows from the first electrode 150 to the fourth electrode 156, an electric field is generated, and voltages are generated at each of the electrodes 150, 152, 154, and 156 based on the electric field. Since the third and fourth electrodes 154 and 156 are covered by the sheath 106, the third voltage V3 178 increases in comparison to when the third electrode 154 is uncovered and the fourth voltage V4 182 increases in comparison to when the fourth electrode 156 is uncovered. In embodiments, to detect the third electrode 154 is covered by the sheath 106, the current I is provided between the first electrode 150 and the third electrode 154, such as sourced by the first electrode 150 and sunk by the third electrode 154, and multiple voltages are measured.

To determine whether the third electrode 154 is covered or uncovered by the sheath 106, two impedance values are determined, one impedance value for each of electrode pair 154 and 150 and electrode pair 154 and 152. In some embodiments, less than two impedance values are determined, such as only one of the three impedance values from electrode pair 154 and 150, electrode pair 154 and 152, or electrode pair 156 and 154. In some embodiments, more than two impedance values are determined, such as all three impedance values from each of electrode pair 154 and 150, electrode pair 154 and 152, and electrode pair 156 and 154.

The voltages at each of the electrodes 150, 152, and 154 are measured and the controller 102 determines the impedance values. A first impedance value is determined by subtracting the first voltage V1 170 from the third voltage V3 178 and dividing the result by a multiple of the current I, and a second impedance value is determined by subtracting the second voltage V2 174 from the third voltage V3 178 and dividing the result by a multiple of the current I. In embodiments, the multiplier “a” is the same for each of the calculations. In some embodiments, the multiplier “a” is different for each of the calculations.

To determine whether the third electrode 154 is covered or uncovered by the sheath 106, each of the two impedance values is compared to a threshold value. In embodiments, the first impedance value is compared to a first threshold value, and the second impedance value is compared to a second threshold value. In some embodiments, the first and second threshold values are the same value. In some embodiments, the first threshold value is different from the second threshold value.

For the ablation system 100 to determine that the third electrode 154 is covered by the sheath 106, the first impedance value must be greater than the first threshold value, and the second impedance value must be greater than the second threshold value.

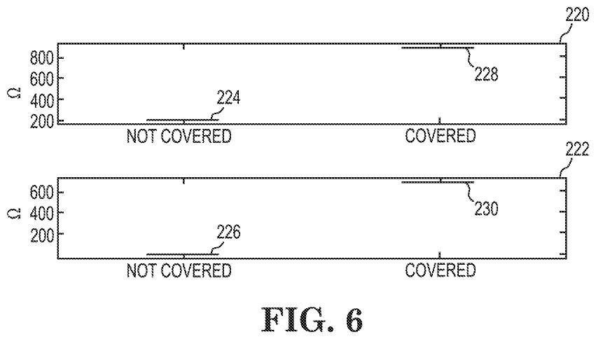

FIG. 6 is a diagram illustrating graphs 220 and 222 of the first and second impedance values, respectively, for determining whether the third electrode 154 is covered by the sheath 106, according to embodiments of the disclosure. Graph 220 shows the first impedance value, determined using the third voltage V3 178 and the first voltage V1 170, with the third electrode 154 uncovered and covered. Graph 222 shows the second impedance value, determined using the third voltage V3 178 and the second voltage V2 174, with the third electrode 154 uncovered and covered. In each of the graphs 220 and 222, the y-axis represents the impedance value in ohms.

The uncovered impedance values 224 and 226 shown in the graphs 220 and 222, respectively, were calculated with the sheath 106 not covering any of the electrodes 150, 152, 154, and 156, as shown in FIG. 2. The voltages at each of the electrodes 150, 152, 154, and 156 were measured and the controller 102 determined the first uncovered impedance value 224 and the second uncovered impedance value 226. The first uncovered impedance value 224 was determined by subtracting the first voltage V1 170 from the third voltage V3 178 and dividing the result by a multiple of the current I and the second uncovered impedance value 226 was determined by subtracting the second voltage V2 174 from the third voltage V3 178 and dividing the result by a multiple of the current I. In embodiments, the multiplier “a” is the same for each of the calculations. In some embodiments, the multiplier “a” is different for each of the calculations.

The covered impedance values 228 and 230 shown in the graphs 220 and 222, respectively, were calculated with the sheath 106 covering the third electrode 154 and the fourth electrode 156 and not covering the other electrodes 150 and 152, as shown in FIG. 5 and as described above in relation to FIG. 5.

The uncovered impedance values 224 and 226 are smaller than the covered impedance values 228 and 230. The first uncovered impedance value 224 is less than or about 200 ohms and the first covered impedance value 228 is greater than 800 ohms, such that a first threshold is set to be between the first uncovered impedance value 224 and the first covered impedance value 228 to distinguish between the uncovered and covered states of the third electrode 154 using the first impedance values 224 and 228. The second uncovered impedance value 226 is less than 200 ohms and the second covered impedance value 230 is greater than 600 ohms, such that a second threshold is set to be between the second uncovered impedance value 226 and the second covered impedance value 230 to distinguish between the uncovered and covered states of the third electrode 154 using the second impedance values 226 and 230.

The uncovered impedance values 224 and 226 of less than or about 200 ohms and less than 200 ohms, respectively, and the covered impedance values 228 and 230 of greater than 800 ohms and greater than 600 ohms, respectively, in the graphs 220 and 222 are example impedance values obtained using one catheter or one type of catheter. In other embodiments, using a different catheter or a different type of catheter, the uncovered impedance values 224 and 226 of less than or about 200 ohms and less than 200 ohms, respectively, may be different values and the covered impedance values 228 and 230 of greater than 800 ohms and greater than 600 ohms, respectively, may be different values and yet still distinguishable from one another, such that thresholds can be set between the uncovered impedance values and the covered impedance values as described above.

The covered impedance values are compared to the threshold values to determine whether the third electrode 154 is uncovered or covered by the sheath 106. In embodiments, all the covered impedance values 228 and 230 must be greater than the corresponding threshold values for the controller 102 to make the determination that the third electrode 154 is covered by the sheath 106. In other embodiments, one or both covered impedance values 228 and 230 must be greater than the corresponding threshold values for the controller 102 to make the determination that the third electrode 154 is covered by the sheath 106.

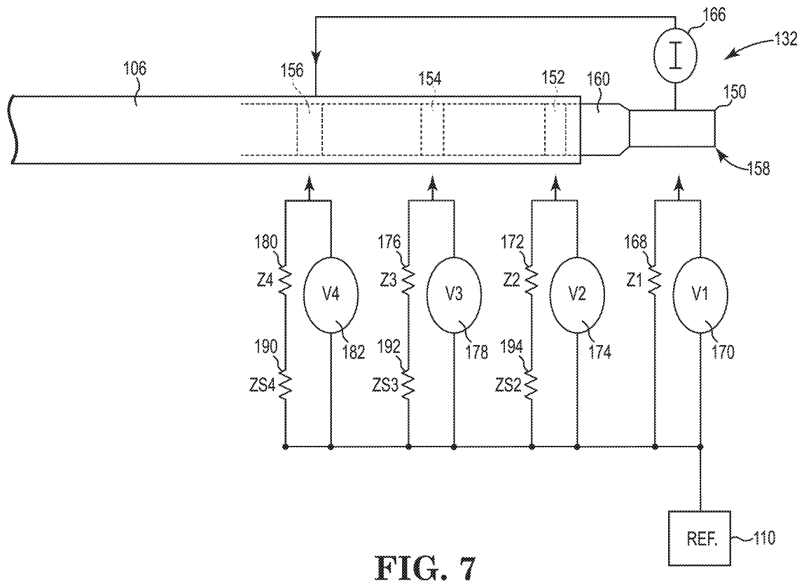

FIG. 7 is a diagram illustrating the sheath 106 covering the fourth electrode 156, the third electrode 154, and the second electrode 152, according to embodiments of the disclosure. In this situation, determining that the second electrode 152 is covered by the sheath 106, also determines that the fourth electrode 156 and the third electrode 154 are covered by the sheath 106.

The distal end 132 of the catheter 104 protrudes from the sheath 106 such that the second, third, and fourth electrodes 152, 154, and 156 are covered by the sheath 106 and the remaining electrode 150 is uncovered or not covered by the sheath 106. The sheath 106 creates a low conducting, high impedance cover over the second, third, and fourth electrodes 152, 154, and 156, which increases the impedance from the second electrode 152 to the reference 110 and the measured second voltage V2 174 on the second electrode 152, increases the impedance from the third electrode 154 to the reference 110 and the measured third voltage V3 178 on the third electrode 154, and which increases the impedance from the fourth electrode 156 to the reference 110 and the measured fourth voltage V4 182 on the fourth electrode 156. The increase in the impedance from the second electrode 152 to the reference 110 is depicted in FIG. 7 with the addition of series impedance ZS2 194 between the second electrode 152 and the reference 110, the increase in the impedance from the third electrode 154 to the reference 110 is depicted with the series resistor ZS3 192 between the third electrode 154 and the reference 110, and the increase in the impedance from the fourth electrode 156 to the reference 110 is depicted with the series resistor ZS4 190 between the fourth electrode 156 and the reference 110.

In operation, the current I at 166 is provided by the controller 102 to the first electrode 150, such that the first electrode 150 sources the current I and the fourth electrode 156 sinks the current I. As the current I at 166 flows from the first electrode 150 to the fourth electrode 156, an electric field is generated, and voltages are generated at each of the electrodes 150, 152, 154, and 156 based on the electric field. Since the second, third, and fourth electrodes 152, 154, and 156 are covered by the sheath 106, the second voltage V2 174 increases in comparison to when the second electrode 152 is uncovered, the third voltage V3 178 increases in comparison to when the third electrode 154 is uncovered, and the fourth voltage V4 182 increases in comparison to when the fourth electrode 156 is uncovered. In embodiments, to detect the second electrode 152 is covered by the sheath 106, the current I is provided between the first electrode 150 and the second electrode 152, such as sourced by the first electrode 150 and sunk by the second electrode 152, and multiple voltages are measured.

To determine whether the second electrode 152 is covered or uncovered by the sheath 106, one impedance value is determined 152 and 150. In some embodiments, more than one impedance value is determined, such as two or all three impedance values from each of electrode pair 152 and 150, electrode pair 152 and 154, and electrode pair 152 and 156.

The voltages at each of the electrodes 150 and 152 are measured and the controller 102 determines the first impedance value by subtracting the first voltage V1 170 from the second voltage V2 174 and dividing the result by a multiple of the current I. To determine whether the second electrode 152 is covered or uncovered by the sheath 106, the first impedance value is compared to a first threshold value. For the ablation system 100 to determine that the second electrode 152 is covered by the sheath 106, the first impedance value must be greater than the first threshold value.

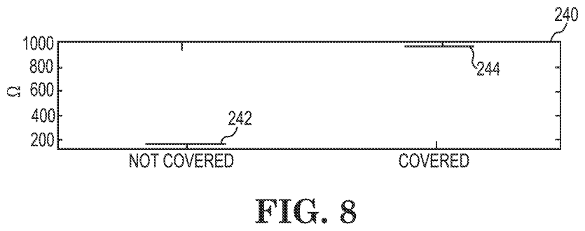

FIG. 8 is a diagram illustrating a graph 240 of the first impedance value for determining whether the second electrode 152 is covered by the sheath 106, according to embodiments of the disclosure. Graph 240 shows the first impedance value, determined using the second voltage V2 174 and the first voltage V1 170, with the second electrode 152 uncovered and covered. In graph 240 the y-axis represents the impedance value in ohms.

The uncovered impedance value 242 shown in the graph 240 was calculated with the sheath 106 not covering any of the electrodes 150, 152, 154, and 156, as shown in FIG. 2. The voltages at each of the electrodes 150, 152, 154, and 156 were measured and the controller 102 determined the first uncovered impedance value 242 by subtracting the first voltage V1 170 from the second voltage V2 174 and dividing the result by a multiple of the current I.

The covered impedance value 244 shown in the graph 240 was calculated with the sheath 106 covering the second electrode 152, the third electrode 154, and the fourth electrode 156 and not covering the first electrodes 150, as shown in FIG. 7 and as described above in relation to FIG. 7.

The uncovered impedance value 242 is smaller than the covered impedance value 244. The first uncovered impedance value 242 is less than or about 200 ohms and the first covered impedance value 244 is greater than 800 ohms, such that a first threshold is set to be between the first uncovered impedance value 242 and the first covered impedance value 244 to distinguish between the uncovered and covered states of the second electrode 152 using the first impedance values 242 and 244.

The uncovered impedance value 242 of less than or about 200 ohms and the covered impedance value 244 of greater than 800 ohms in the graph 240 are example impedance values obtained using one catheter or one type of catheter. In other embodiments, using a different catheter or a different type of catheter, the uncovered impedance value 242 of less than or about 200 ohms and the covered impedance value 244 of greater than 800 ohms may be different values and yet still distinguishable from one another, such that a threshold can be set between the uncovered impedance value and the covered impedance value as described above.

The covered impedance value is compared to the threshold value to determine whether the second electrode 152 is uncovered or covered by the sheath 106. In embodiments, the covered impedance value 244 must be greater than the corresponding threshold value for the controller 102 to make the determination that the second electrode 152 is covered by the sheath 106.

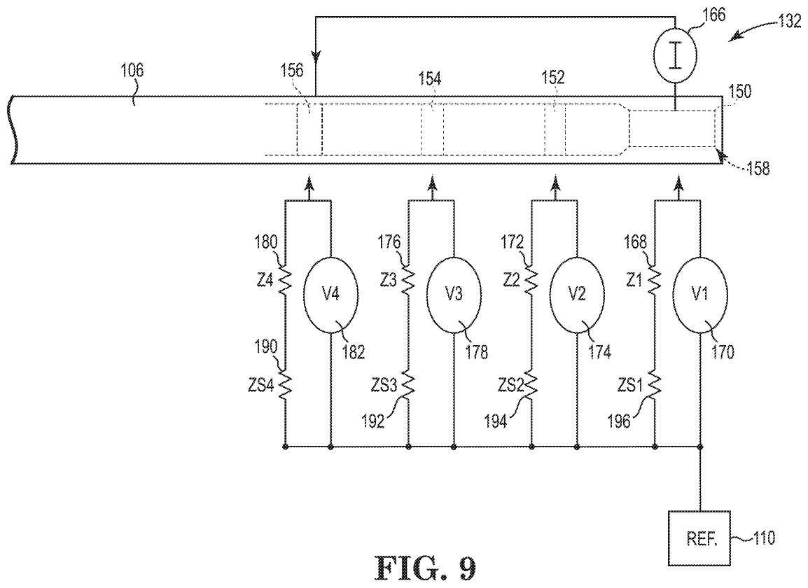

FIG. 9 is a diagram illustrating the sheath 106 covering the fourth electrode 156, the third electrode 154, the second electrode 152, and the first electrode 150, according to embodiments of the disclosure. In this situation, determining that the first electrode 150 is covered by the sheath 106, also determines that the fourth electrode 156, the third electrode 154, and the second electrode 152 are covered by the sheath 106.

The distal end 132 of the catheter 104 protrudes from the sheath 106 such that the first, second, third, and fourth electrodes 150, 152, 154, and 156 are covered by the sheath 106. The sheath 106 creates a low conducting, high impedance cover over the first, second, third, and fourth electrodes 150, 152, 154, and 156, which increases the impedance from the first electrode 150 to the reference 110 and the measured first voltage V1 170 on the first electrode 150, increases the impedance from the second electrode 152 to the reference 110 and the measured second voltage V2 174 on the second electrode 152, increases the impedance from the third electrode 154 to the reference 110 and the measured third voltage V3 178 on the third electrode 154, and which increases the impedance from the fourth electrode 156 to the reference 110 and the measured fourth voltage V4 182 on the fourth electrode 156. The increase in the impedance from the first electrode 150 to the reference 110 is depicted in FIG. 9 with the addition of series impedance ZS1 196 between the first electrode 150 and the reference 110, the increase in the impedance from the second electrode 152 to the reference 110 is depicted with the series impedance ZS2 194 between the second electrode 152 and the reference 110, the increase in the impedance from the third electrode 154 to the reference 110 is depicted with the series impedance ZS3 192 between the third electrode 154 and the reference 110, and the increase in the impedance from the fourth electrode 156 to the reference 110 is depicted with the series impedance ZS4 190 between the fourth electrode 156 and the reference 110.

In operation, the current I at 166 is provided by the controller 102 to the first electrode 150, such that the first electrode 150 sources the current I and the fourth electrode 156 sinks the current I. As the current I at 166 flows from the first electrode 150 to the fourth electrode 156, an electric field is generated, and voltages are generated at each of the electrodes 150, 152, 154, and 156 based on the electric field. Since the first, second, third, and fourth electrodes 150, 152, 154, and 156 are covered by the sheath 106, the first voltage V1 170 increases in comparison to when the first electrode 150 is uncovered, the second voltage V2 174 increases in comparison to when the second electrode 152 is uncovered, the third voltage V3 178 increases in comparison to when the third electrode 154 is uncovered, and the fourth voltage V4 182 increases in comparison to when the fourth electrode 156 is uncovered. In embodiments, to detect the first electrode 150 is covered by the sheath 106, the current I is provided between the first electrode 150 and any one or more of the other electrodes 152, 154, and 156, such as sourced by the first electrode 150 and sunk by any one of the other electrodes 152, 154, and 156, and multiple voltages are measured.

To determine whether the first electrode 150 is covered or uncovered by the sheath 106, three impedance values are determined, one impedance value for each of electrode pair 150 and 156, electrode pair 150 and 154, and electrode pair 150 and 152. In some embodiments, less than three impedance values are determined, such as any one or two impedance values from each of electrode pair 150 and 156, electrode pair 150 and 154, and electrode pair 150 and 152.

The voltages at each of the electrodes 150, 152, 154, and 156 are measured and the controller 102 determines the three impedance values. With the first electrode 150 covered by the sheath 106, the first voltage V1 170 goes up very high, such as up to 10 times higher than the other voltages V2, V3, and V4. A first impedance value is determined by subtracting the second voltage V2 174 from the first voltage V1 170 and dividing the result by a multiple of the current I. A second impedance value is determined by subtracting the third voltage V3 178 from the first voltage V1 170 and dividing the result by a multiple of the current I. A third impedance value is determined by subtracting the fourth voltage V4 182 from the first voltage V1 170 and dividing the result by a multiple of the current I. In embodiments, the multiplier “a” is the same for each of the three calculations. In some embodiments, the multiplier “a” is different for one or more of the three calculations.

To determine whether the first electrode 150 is covered or uncovered by the sheath 106, each of the three impedance values is compared to a threshold value. In embodiments, the first impedance value is compared to a first threshold value, the second impedance value is compared to a second threshold value, and the third impedance value is compared to a third threshold value. In some embodiments, the first, second, and third threshold values are the same value. In some embodiments, one or more of the first, second, and third threshold values are different from the other threshold value(s).

For the ablation system 100 to determine that the first electrode 150 is covered by the sheath 106, the first impedance value must be greater than the first threshold value, the second impedance value must be greater than the second threshold value, and the third impedance value must be greater than the third threshold value.

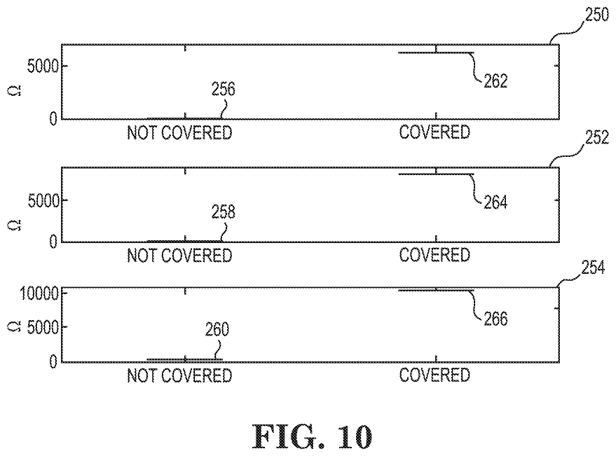

FIG. 10 is a diagram illustrating graphs 250, 252, and 254 of the first, second, and third impedance values, respectively, according to embodiments of the disclosure. Graph 250 shows the first impedance value, determined using the first voltage V1 170 and the second voltage V2 174, with the first electrode 150 uncovered and covered. Graph 252 shows the second impedance value, determined using the first voltage V1 170 and the third voltage V3 178, with the first electrode 150 uncovered and covered. Graph 254 shows the third impedance value, determined using the first voltage V1 170 and the fourth voltage V4 182, with the first electrode 150 uncovered and covered. In each of the graphs 250, 252, and 254, the y-axis represents the impedance value in ohms.

The uncovered impedance values 256, 258, and 260 shown in the graphs 250, 252, and 254, respectively, were calculated with the sheath 106 not covering any of the electrodes 150, 152, 154, and 156, as shown in FIG. 2. The voltages at each of the electrodes 150, 152, 154, and 156 were measured and the controller 102 determined the first uncovered impedance value 256, the second uncovered impedance value 258, and the third uncovered impedance value 260. The first uncovered impedance value 256 was determined by subtracting the second voltage V2 174 from the first voltage V1 170 and dividing the result by a multiple of the current I. The second uncovered impedance value 258 was determined by subtracting the third voltage V3 178 from the first voltage V1 170 and dividing the result by a multiple of the current I. The third uncovered impedance value 260 was determined by subtracting the fourth voltage V4 182 from the first voltage V1 170 and dividing the result by a multiple of the current I. In embodiments, the multiplier “a” is the same for each of the three calculations. In some embodiments, the multiplier “a” is different for one or more of the three calculations.

The covered impedance values 262, 264, and 266 shown in the graphs 250, 252, and 254, respectively, were calculated with the sheath 106 covering the first electrode 150 and all other electrodes 152, 154, and 156, as shown in FIG. 9 and as described above in relation to FIG. 9.

The uncovered impedance values 256, 258, and 260 are smaller than the covered impedance values 262, 264, and 266. The first uncovered impedance value 256 is less than 1000 ohms and closer to zero ohms and the first covered impedance value 262 is greater than 5000 ohms, such that a first threshold is set to be between the first uncovered impedance value 256 and the first covered impedance value 262 to distinguish between the uncovered and covered states of the first electrode 150 using the first impedance values 256 and 262. The second uncovered impedance value 258 is less than 1000 ohms and closer to zero ohms and the second covered impedance value 264 is greater than 5000 ohms, such that a second threshold is set to be between the second uncovered impedance value 258 and the second covered impedance value 264 to distinguish between the uncovered and covered states of the first electrode 150 using the second impedance values 258 and 264. The third uncovered impedance value 260 is less than 1000 ohms and closer to zero ohms and the third covered impedance value 262 is greater than or close to 10,000 ohms, such that a third threshold is set to be between the third uncovered impedance value 260 and the third covered impedance value 266 to distinguish between the uncovered and covered states of the first electrode 150 using the third impedance values 260 and 266.

The uncovered impedance values 256, 258, and 260 of less than 1000 ohms and the covered impedance values 262 and 264 of greater than 5000 ohms and 266 of greater than or close to 10,000 ohms in the graphs 250, 252, and 254 are example impedance values obtained using one catheter or one type of catheter. In other embodiments, using a different catheter or a different type of catheter, the uncovered impedance values 256, 258, and 260 of less than 1000 ohms may be different values and the covered impedance values 262 and 264 of greater than 5000 ohms and 266 of greater than or close to 10,000 ohms may be different values and yet still distinguishable from one another, such that thresholds can be set between the uncovered impedance values and the covered impedance values as described above.

The covered impedance values are compared to the threshold values to determine whether the first electrode 150 is uncovered or covered by the sheath 106. In embodiments, all the covered impedance values 262, 264, and 266 must be greater than the corresponding threshold values for the controller 102 to make the determination that the first electrode 150 is covered by the sheath 106. In other embodiments, one or more of the covered impedance values 262, 264, and 266 must be greater than the corresponding threshold values for the controller 102 to make the determination that the first electrode 150 is covered by the sheath 106.

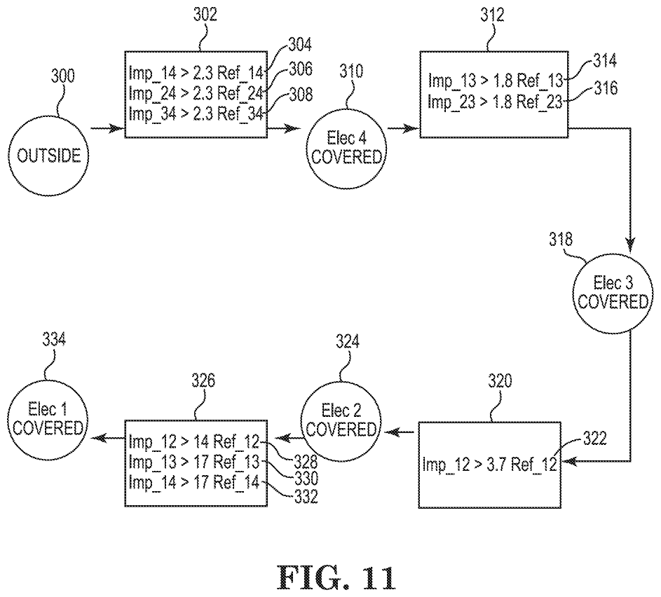

FIG. 11 is a diagram illustrating a method of determining which, if any, of the first, second, third, and fourth electrodes 150, 152, 154, and 156 are covered by the sheath 106, according to embodiments of the disclosure. In embodiments, the method includes moving the sheath 106 from not covering any of the electrodes 150, 152, 154, and 156 to covering all the electrodes 150, 152, 154, and 156.

In this method, thresholds are calculated by multiplying constants, such as 2.3, 1.8, 3.7, 14, and 17, times uncovered impedance values. The values of the constants are example constant values that can be used to calculate thresholds in one embodiment of the method, such as for one catheter or one type of catheter. In other embodiments, including embodiments that use a different catheter or a different type of catheter, the constants can be different values.

At 300, the sheath 106 is not covering any of the electrodes 150, 152, 154, and 156. The conditions or tests at 302, determine whether the fourth electrode 156 is covered by the sheath 106. The conditions 302 include first, second, and third equations 304, 306, and 308 for determining whether the fourth electrode 156 is covered. In embodiments, the controller 102 determines that the fourth electrode 156 is covered by the sheath 106 if each of, and all three of, the first, second, and third equations 304, 306, and 308 is satisfied in the affirmative. In other embodiments, the controller 102 may determine that the fourth electrode 156 is covered by the sheath 106 if only one or two of the first, second, and third equations 304, 306, and 308 is satisfied in the affirmative.

The first, second, and third calculated impedance values, indicated by Imp_14, Imp_24, and Imp_34, respectively, and the first, second, and third uncovered impedance values, indicated by Ref_14, Ref_24, and Ref_34, respectively, are calculated and determined as described above in the description of FIGS. 3 and 4. In the first equation 304, the calculated first impedance value Imp_14 is compared to the first threshold of 2.3 multiplied times the uncovered first impedance value of Ref_14. If the calculated first impedance value Imp_14 is greater than the first threshold, the condition is satisfied in the affirmative. In the second equation 306, the calculated second impedance value Imp_24 is compared to the second threshold of 2.3 multiplied times the uncovered second impedance value of Ref_24. If the calculated second impedance value Imp_24 is greater than the second threshold, the condition is satisfied in the affirmative. In the third equation 308, the calculated third impedance value Imp_34 is compared to the third threshold of 2.3 multiplied times the uncovered third impedance value of Ref_34. If the calculated third impedance value Imp_34 is greater than the third threshold, the condition is satisfied in the affirmative. At 310, if all three of the first, second, and third equations 304, 306, and 308 are satisfied in the affirmative, the controller 102 makes the determination that the fourth electrode 156 is covered by the sheath 106.

The conditions or tests at 312, determine whether the third electrode 154 is covered by the sheath 106. The conditions 312 include first and second equations 314 and 316 for determining whether the third electrode 154 is covered. In embodiments, the controller 102 determines that the third electrode 154 is covered by the sheath 106 if each of, and both of, the first and second equations 314 and 316 is satisfied in the affirmative. In other embodiments, the controller 102 may determine that the third electrode 154 is covered by the sheath 106 if only one of the two equations 314 and 316 is satisfied in the affirmative.

The first and second calculated impedance values, indicated by Imp_13 and Imp_23, respectively, and the first and second uncovered impedance values, indicated by Ref_13 and Ref_23, respectively, are calculated and determined as described above in the description of FIGS. 5 and 6. In the first equation 314, the calculated first impedance value Imp_13 is compared to the first threshold of 1.8 multiplied times the uncovered first impedance value of Ref_13. If the calculated first impedance value Imp_13 is greater than the first threshold, the condition is satisfied in the affirmative. In the second equation 316, the calculated second impedance value Imp_23 is compared to the second threshold of 1.8 multiplied times the uncovered second impedance value of Ref_23. If the calculated second impedance value Imp_23 is greater than the second threshold, the condition is satisfied in the affirmative. At 318, if both first and second equations 314 and 316 are satisfied in the affirmative, the controller 102 makes the determination that the third electrode 154 is covered by the sheath 106.

The condition or test at 320, determines whether the second electrode 152 is covered by the sheath 106. The condition 320 includes a first equation 322 for determining whether the second electrode 152 is covered. In embodiments, the controller 102 determines that the second electrode 152 is covered by the sheath 106 if the first equation 322 is satisfied in the affirmative. In other embodiments, the controller 102 may determine that the second electrode 152 is covered by the sheath 106 using other equations and calculated impedance values.

The first calculated impedance value, indicated by Imp_12, and the first uncovered impedance value, indicated by Ref_12, are calculated and determined as described above in the description of FIGS. 7 and 8. In the first equation 322, the calculated first impedance value Imp_12 is compared to the first threshold of 3.7 multiplied times the uncovered first impedance value of Ref_12. If the calculated first impedance value Imp_12 is greater than the first threshold, the condition is satisfied in the affirmative. At 324, if the first equation 322 is satisfied in the affirmative, the controller 102 makes the determination that the second electrode 152 is covered by the sheath 106.

The conditions or tests at 326, determine whether the first electrode 150, and consequently all the electrodes 150, 152, 154, and 156, is covered by the sheath 106. The conditions 326 include first, second, and third equations 328, 330, and 332 for determining whether the first electrode 150 is covered. In embodiments, the controller 102 determines that the first electrode 150 is covered by the sheath 106 if each of, and all three of, the first, second, and third equations 328, 330, and 332 is satisfied in the affirmative. In other embodiments, the controller 102 may determine that the first electrode 150 is covered by the sheath 106 if only one or two of the first, second, and third equations 328, 330, and 332 is satisfied in the affirmative.

The first, second, and third calculated impedance values, indicated by Imp_12, Imp_13, and Imp_14, respectively, and the first, second, and third uncovered impedance values, indicated by Ref_12, Ref_13, and Ref_14, respectively, are calculated and determined as described above in the description of FIGS. 9 and 10. In the first equation 328, the calculated first impedance value Imp_12 is compared to the first threshold of 14 multiplied times the uncovered first impedance value of Ref_12. If the calculated first impedance value Imp_12 is greater than the first threshold, the condition is satisfied in the affirmative. In the second equation 330, the calculated second impedance value Imp_13 is compared to the second threshold of 17 multiplied times the uncovered second impedance value of Ref_13. If the calculated second impedance value Imp_13 is greater than the second threshold, the condition is satisfied in the affirmative. In the third equation 332, the calculated third impedance value Imp_14 is compared to the third threshold of 17 multiplied times the uncovered third impedance value of Ref_14. If the calculated third impedance value Imp_14 is greater than the third threshold, the condition is satisfied in the affirmative. At 334, if all three of the first, second, and third equations 328, 330, and 332 are satisfied in the affirmative, the controller 102 makes the determination that the first electrode 150 is covered by the sheath 106.

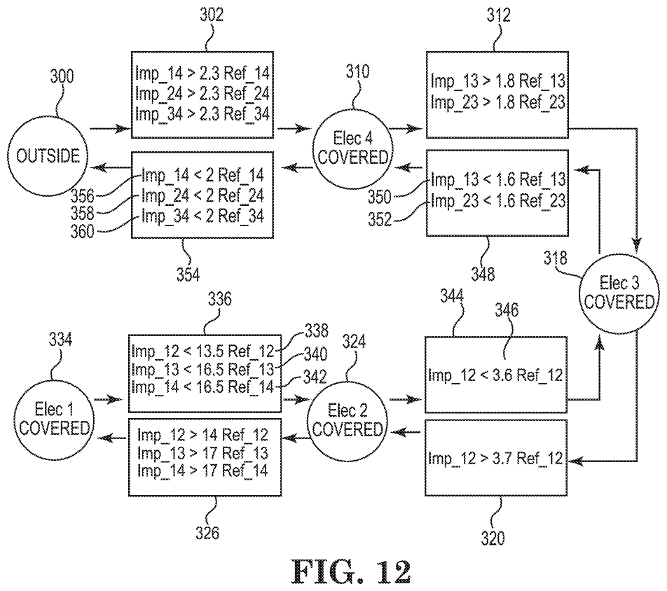

FIG. 12 is a diagram further illustrating a method of determining which, if any, of the first, second, third, and fourth electrodes 150, 152, 154, and 156 are covered by the sheath 106, according to embodiments of the disclosure. In embodiments, the method includes moving the sheath 106 from covering all the electrodes 150, 152, 154, and 156 at 334 to not covering any of the electrodes 150, 152, 154, and 156 at 300.

In this method, thresholds are calculated by multiplying constants times uncovered impedance values. The values of the constants are example constant values that can be used to calculate thresholds in one embodiment of the method, or for one catheter or one type of catheter. In other embodiments, including embodiments that use a different catheter or a different type of catheter, the constants can be different values.

At 334, the sheath 106 covers all the electrodes 150, 152, 154, and 156. The conditions or tests at 336, determine whether the first electrode 150 is uncovered by the sheath 106. The conditions 336 include first, second, and third equations 338, 340, and 342 for determining whether the first electrode 150 is uncovered. In embodiments, the controller 102 determines that the first electrode 150 is uncovered by the sheath 106 if each of, and all three of, the first, second, and third equations 338, 340, and 342 is satisfied in the affirmative. In other embodiments, the controller 102 may determine that the first electrode 150 is uncovered by the sheath 106 if only one or two of the first, second, and third equations 338, 340, and 342 is satisfied in the affirmative.

The first, second, and third calculated impedance values, indicated by Imp_12, Imp_13, and Imp_14, respectively, and the first, second, and third uncovered impedance values, indicated by Ref_12, Ref_13, and Ref_14, respectively, are calculated and determined as described above in the description of FIGS. 9 and 10. In the first equation 338, the calculated first impedance value Imp_12 is compared to the first threshold of 13.5 multiplied times the uncovered first impedance value of Ref_12. If the calculated first impedance value Imp_12 is less than the first threshold, the condition is satisfied in the affirmative. In the second equation 340, the calculated second impedance value Imp_13 is compared to the second threshold of 16.5 multiplied times the uncovered second impedance value of Ref 13. If the calculated second impedance value Imp_13 is less than the second threshold, the condition is satisfied in the affirmative. In the third equation 342, the calculated third impedance value Imp_14 is compared to the third threshold of 16.5 multiplied times the uncovered third impedance value of Ref 14. If the calculated third impedance value Imp_14 is less than the third threshold, the condition is satisfied in the affirmative. At 324, if all three of the first, second, and third equations 328, 330, and 332 are satisfied in the affirmative, the controller 102 makes the determination that the first electrode 150 is uncovered by the sheath 106.

The condition or test at 344, determines whether the second electrode 152 is uncovered by the sheath 106. The condition 344 includes a first equation 346 for determining whether the second electrode 152 is covered. In embodiments, the controller 102 determines that the second electrode 152 is uncovered by the sheath 106 if the first equation 346 is satisfied in the affirmative. In other embodiments, the controller 102 may determine that the second electrode 152 is uncovered by the sheath 106 using other equations and calculated impedance values.

The first calculated impedance value, indicated by Imp_12, and the first uncovered impedance value, indicated by Ref_12, are calculated and determined as described above in the description of FIGS. 7 and 8. In the first equation 344, the calculated first impedance value Imp_12 is compared to the first threshold of 3.6 multiplied times the uncovered first impedance value of Ref_12. If the calculated first impedance value Imp_12 is less than the first threshold, the condition is satisfied in the affirmative. At 318, if the first equation 346 is satisfied in the affirmative, the controller 102 makes the determination that the second electrode 152 is uncovered by the sheath 106.

The conditions or tests at 348, determine whether the third electrode 154 is uncovered by the sheath 106. The conditions 348 include first and second equations 350 and 352 for determining whether the third electrode 154 is uncovered. In embodiments, the controller 102 determines that the third electrode 154 is uncovered by the sheath 106 if each of, and both of, the first and second equations 350 and 352 is satisfied in the affirmative. In other embodiments, the controller 102 may determine that the third electrode 154 is uncovered by the sheath 106 if only one of the two equations 350 and 352 is satisfied in the affirmative.

The first and second calculated impedance values, indicated by Imp_13 and Imp_23, respectively, and the first and second uncovered impedance values, indicated by Ref_13 and Ref_23, respectively, are calculated and determined as described above in the description of FIGS. 5 and 6. In the first equation 350, the calculated first impedance value Imp_13 is compared to the first threshold of 1.6 multiplied times the uncovered first impedance value of Ref_13. If the calculated first impedance value Imp_13 is less than the first threshold, the condition is satisfied in the affirmative. In the second equation 352, the calculated second impedance value Imp_23 is compared to the second threshold of 1.6 multiplied times the uncovered second impedance value of Ref_23. If the calculated second impedance value Imp_23 is less than the second threshold, the condition is satisfied in the affirmative. At 310, if both first and second equations 350 and 352 are satisfied in the affirmative, the controller 102 makes the determination that the third electrode 154 is uncovered by the sheath 106.

At 300, the sheath 106 is not covering any of the electrodes 150, 152, 154, and 156. The conditions or tests at 354, determine whether the fourth electrode 156 is uncovered by the sheath 106. The conditions 354 include first, second, and third equations 356, 358, and 360 for determining whether the fourth electrode 156 is uncovered. In embodiments, the controller 102 determines that the fourth electrode 156 is uncovered by the sheath 106 if each of, and all three of, the first, second, and third equations 356, 358, and 360 is satisfied in the affirmative. In other embodiments, the controller 102 may determine that the fourth electrode 156 is uncovered by the sheath 106 if only one or two of the first, second, and third equations 356, 358, and 360 is satisfied in the affirmative.

The first, second, and third calculated impedance values, indicated by Imp_14, Imp_24, and Imp_34, respectively, and the first, second, and third uncovered impedance values, indicated by Ref_14, Ref_24, and Ref_34, respectively, are calculated and determined as described above in the description of FIGS. 3 and 4. In the first equation 356, the calculated first impedance value Imp_14 is compared to the first threshold of 2.0 multiplied times the uncovered first impedance value of Ref_14. If the calculated first impedance value Imp_14 is less than the first threshold, the condition is satisfied in the affirmative. In the second equation 358, the calculated second impedance value Imp_24 is compared to the second threshold of 2.0 multiplied times the uncovered second impedance value of Ref_24. If the calculated second impedance value Imp_24 is less than the second threshold, the condition is satisfied in the affirmative. In the third equation 360, the calculated third impedance value Imp_34 is compared to the third threshold of 2.0 multiplied times the uncovered third impedance value of Ref 34. If the calculated third impedance value Imp_34 is less than the third threshold, the condition is satisfied in the affirmative. At 300, if all three of the first, second, and third equations 356, 358, and 360 are satisfied in the affirmative, the controller 102 makes the determination that the fourth electrode 156 is uncovered by the sheath 106.

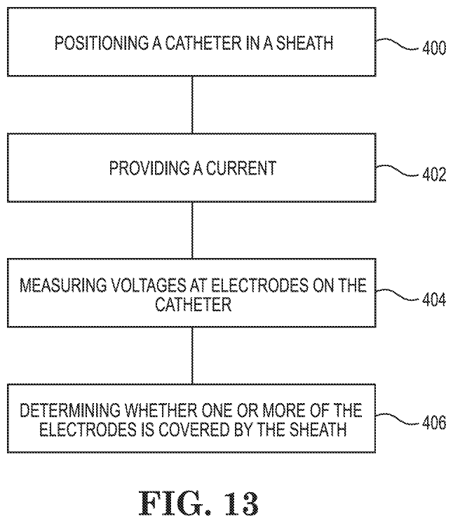

FIG. 13 is a diagram illustrating a method for determining whether one or more electrodes on a catheter are covered by a sheath, and for determining the location of the sheath on the catheter, according to embodiments of the disclosure. In embodiments, the method includes determining whether one or more electrodes 150, 152, 154, and 156 on catheter 104 are covered by sheath 106 as described above, and for determining the location of the sheath 106 on the catheter 104.

The method, at 400, includes positioning the distal end of the catheter through a lumen of the sheath. The distal end of the catheter includes electrodes configured to protrude from a distal end of the sheath. The sheath is configured to cover the electrodes of the catheter.

At 402, the method includes providing a current between electrodes at the distal end of the catheter. In embodiments, providing a current comprises sourcing the current from one of the electrodes and sinking the current at another one of the electrodes. In embodiments, providing a current comprises sourcing the current from a most distal one of the electrodes and sinking the current at a most proximal one of the electrodes.

At 404, the method includes measuring voltages from each of at least two of the electrodes to a reference voltage and, at 406, the method includes determining whether one or more of the electrodes is covered by the sheath based on the current and the voltages measured. In embodiments, determining whether one or more of the electrodes is covered by the sheath includes calculating at least one impedance value, and comparing the at least one impedance value to one or more threshold values. In embodiments, determining whether one or more of the electrodes is covered by the sheath comprises calculating at least one impedance value by determining a difference between two of the voltages, and dividing the difference by a multiple of the current.

In embodiments, the method includes positioning a sheath over a catheter that includes four electrodes spaced apart at a distal end of the catheter. The catheter includes a first electrode that is a most distal electrode at the distal end of the catheter, a second electrode spaced apart from and proximal the first electrode, a third electrode spaced apart from and proximal the second electrode, and a fourth electrode spaced apart from and proximal the third electrode. The distal end of the catheter is configured to protrude from a distal end of the sheath that is configured to cover at least up to all four of the four electrodes. This method further includes providing a current between any two of the four electrodes, measuring voltages from at least two of the four electrodes to a reference voltage, and determining whether one or more of the four electrodes is covered by the sheath.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical system, comprising:
a catheter having a catheter distal end and including multiple electrodes situated at the catheter distal end;
a sheath having a sheath distal end and a lumen configured to receive the catheter such that the catheter distal end protrudes from the sheath distal end, the sheath configured to cover one or more of the multiple electrodes; and
a controller configured to provide a current between electrodes of the multiple electrodes and measure at least two voltages from at least two of the multiple electrodes to a reference voltage to determine whether one or more of the multiple electrodes is covered by the sheath.

2. The medical system of claim 1, wherein the controller is configured to source the current from any one of the multiple electrodes and sink the current at any other one of the multiple electrodes.

3. The medical system of claim 1, wherein the controller is configured to measure a voltage from each of the multiple electrodes to the reference voltage to determine whether the one or more of the multiples electrodes is covered by the sheath.

4. The medical system of claim 1, wherein the controller is configured to calculate impedance values between electrodes of the multiple electrodes to determine whether the one or more of the multiple electrodes is covered by the sheath.

5. The medical system of claim 4, wherein the controller is configured to compare the impedance values calculated to one or more threshold values to determine whether the one or more of the multiple electrodes is covered by the sheath.

6. The medical system of claim 1, wherein the controller is configured to calculate impedance values between electrodes of the multiple electrodes by determining a difference between the at least two voltages measured and dividing the difference by a multiple of the current provided via the controller.

7. The medical system of claim 1, wherein the multiple electrodes are longitudinally spaced apart at the catheter distal end.

8. The medical system of claim 1, wherein the catheter is a linear ablation catheter.

9. A method, comprising:
positioning a distal end of a catheter through a lumen of a sheath that is configured to cover electrodes at the distal end of the catheter, the distal end of the catheter including the electrodes configured to protrude from a distal end of the sheath;
providing a current between electrodes at the distal end of the catheter;
measuring voltages from each of at least two of the electrodes to a reference voltage; and
determining whether one or more of the electrodes is covered by the sheath based on the current and the voltages measured.

10. The method of claim 9, wherein providing a current comprises sourcing the current from one of the electrodes and sinking the current at another one of the electrodes.

11. The method of claim 9, wherein providing a current comprises sourcing the current from a most distal one of the electrodes and sinking the current at a most proximal one of the electrodes.

12. The method of claim 9, wherein determining whether one or more of the electrodes is covered by the sheath comprises:
calculating at least one impedance value; and
comparing the at least one impedance value to one or more threshold values.

13. The method of claim 9, wherein determining whether one or more of the electrodes is covered by the sheath comprises calculating at least one impedance value by;
determining a difference between two of the voltages; and
dividing the difference by a multiple of the current.

14. A method, comprising:
positioning a sheath over a catheter that includes four electrodes spaced apart at a distal end of the catheter, the catheter including a first electrode that is a most distal electrode at the distal end of the catheter, a second electrode spaced apart from and proximal the first electrode, a third electrode spaced apart from and proximal the second electrode, and a fourth electrode spaced apart from and proximal the third electrode, the distal end of the catheter configured to protrude from a distal end of the sheath that is configured to cover at least up to all four of the four electrodes;
providing a current between any two of the four electrodes;
measuring voltages from at least two of the four electrodes to a reference voltage; and
determining whether one or more of the four electrodes is covered by the sheath.

15. The method of claim 14, wherein providing a current comprises sourcing the current from the first electrode and sinking the current at the fourth electrode.

16. The method of claim 14, wherein to determine whether the fourth electrode is covered by the sheath:
measuring voltages from at least two of the four electrodes comprises measuring voltages from the first electrode to the reference voltage to get a first voltage, from the second electrode to the reference voltage to get a second voltage, from the third electrode to the reference voltage to get a third voltage, and from the fourth electrode to the reference voltage to get a fourth voltage; and
determining whether one or more of the four electrodes is covered by the sheath comprises:
subtracting the first voltage from the fourth voltage to get a first result and dividing the first result by a first multiple of the current to get a first impedance value;
subtracting the second voltage from the fourth voltage to get a second result and dividing the second result by a second multiple of the current to get a second impedance value;
subtracting the third voltage from the fourth voltage to get a third result and dividing the third result by a third multiple of the current to get a third impedance value;
comparing the first impedance value to a first threshold value;
comparing the second impedance value to a second threshold value; and
comparing the third impedance value to a third threshold value.

17. The method of claim 14, wherein to determine whether the third electrode is covered by the sheath:
measuring voltages from at least two of the four electrodes comprises measuring voltages from the first electrode to the reference voltage to get a first voltage, from the second electrode to the reference voltage to get a second voltage, and from the third electrode to the reference voltage to get a third voltage; and
determining whether one or more of the four electrodes is covered by the sheath comprises:
subtracting the first voltage from the third voltage to get a first result and dividing the first result by a first multiple of the current to get a first impedance value;
subtracting the second voltage from the third voltage to get a second result and dividing the second result by a second multiple of the current to get a second impedance value;
comparing the first impedance value to a first threshold value; and
comparing the second impedance value to a second threshold value.

18. The method of claim 14, wherein to determine whether the second electrode is covered by the sheath:
measuring voltages from at least two of the four electrodes comprises measuring voltages from the first electrode to the reference voltage to get a first voltage, and from the second electrode to the reference voltage to get a second voltage; and
determining whether one or more of the four electrodes is covered by the sheath comprises:
subtracting the first voltage from the second voltage to get a first result and dividing the first result by a first multiple of the current to get a first impedance value; and
comparing the first impedance value to a first threshold value.

19. The method of claim 14, wherein to determine whether the first electrode is covered by the sheath:
measuring voltages from at least two of the four electrodes comprises measuring voltages from the first electrode to the reference voltage to get a first voltage, from the second electrode to the reference voltage to get a second voltage, from the third electrode to the reference voltage to get a third voltage, and from the fourth electrode to the reference voltage to get a fourth voltage; and
determining whether one or more of the four electrodes is covered by the sheath comprises:
subtracting the second voltage from the first voltage to get a first result and dividing the first result by a first multiple of the current to get a first impedance value;
subtracting the third voltage from the first voltage to get a second result and dividing the second result by a second multiple of the current to get a second impedance value;
subtracting the fourth voltage from the first voltage to get a third result and dividing the third result by a third multiple of the current to get a third impedance value;
comparing the first impedance value to a first threshold value;
comparing the second impedance value to a second threshold value; and
comparing the third impedance value to a third threshold value.

20. The method of claim 14, wherein positioning a sheath over a catheter comprises uncovering one or more of the four electrodes and covering up to all four of the four electrodes.
